(19) United States
(12) Patent Application Publication
(10) Pub. No.: US 2022/0054147 A1
(43) Pub. Date: Feb. 24, 2022

(54) Intraosseous Access System To Automatically Detect Medullary Cavity

(71) Applicant: Bard Access Systems, Inc., Salt Lake City, UT (US)

(72) Inventor: Robin Scott Urry, Layton, UT (US)

(21) Appl. No.: 17/405,692

(22) Filed: Aug. 18, 2021

Related U.S. Application Data
(60) Provisional application No. 63/067,754, filed on Aug. 19, 2020.

(51) Int. Cl. A61B 17/16; A61M 39/02; G16H 40/67; G05B 19/4155

(52) U.S. Cl. CPC A61B 17/1626; A61M 39/0247; A61B 17/1628; A61B 2017/00039; G05B 19/4155; A61M 2039/025; A61M 2039/0291; G16H 40/67

(57) ABSTRACT

An intraosseous access system to access a medullary cavity includes a driver including an access assembly, a motor, and an energy source. The intraosseous access system further includes a sensor configured to detect a first input from one of the motor or the energy source. The intraosseous access system further including a processing unit, communicatively coupled with the sensor, configured to receive the first input from the sensor, and determine access to a medullary cavity. The processing unit can then modify operation of one of the motor and the energy source to automatically stop operation of the system and prevent backwalling.

### PRIORITY

This application claims the benefit of priority to U.S. Provisional Application No. 63/067,754, filed Aug. 19, 2020, which is incorporated by reference in its entirety into this application.

### BACKGROUND

Intraosseous (“IO”) access devices often require training to ensure correct placement of the access device. Users must apply sufficient longitudinal driving force to penetrate the bone without applying too much driving force that can result in “back walling,” where a needle penetrates a far wall of the bone. Further complications can arise when accessing bones of different sizes and density, depending on the age and health of the patient. Moreover, IO devices are often used in emergency situations where delays can be critical and fully trained users may not always be available. Thus, having an IO device that could automatically detect when the user has accessed the medullary cavity, and stop the IO device from further distal advancement would allow users with little to no training to place access devices rapidly and reduce the risk of back walling. Embodiments disclosed herein are directed to intraosseous (IO) access devices including sensors and processing units that are configured to automatically detect access to a medullary cavity and modify the activation of the drill.

### SUMMARY

Disclosed herein is intraosseous access system including, in some embodiments, a driver including an access assembly, a motor, and an energy source, a sensor configured to detect a first input from one of the motor or the energy source, and a processing unit, communicatively coupled with the sensor, configured to receive the first input from the sensor and determine access to a medullary cavity and to modify operation of one of the motor and the energy source.

In some embodiments, the intraosseous access system includes where the processing unit compares a second input relative to the first input to determine access to a medullary cavity.

In some embodiments, the intraosseous access system includes where the processing unit compares the first input relative to a threshold value to determine access to a medullary cavity.

In some embodiments, the intraosseous access system includes where the sensor is configured to detect one of an electrical measurement or a mechanical measurement.

In some embodiments, the intraosseous access system includes where the sensor includes one of an ammeter, ohmmeter, voltmeter, torque meter, or tachometer.

In some embodiments, the intraosseous access system includes where the sensor and the processing unit are located within the driver.

In some embodiments, the intraosseous access system includes where the sensor is located within the driver and the processing unit is located remotely from the driver and is in wireless communication with one of the sensor, the motor, or the energy source.

In some embodiments, the intraosseous access system includes where the threshold value is a predetermined value.

In some embodiments, the intraosseous access system includes where the threshold value can be derived by the processing unit relative to one of the first input or a second input.

In some embodiments, the intraosseous access system includes where the threshold value can be calibrated for electrical current draws required to drive the intraosseous access system through tissues to access the medullary cavity.

In some embodiments, the intraosseous access system includes where the threshold value can be normalized for differences in age, sex, health condition of a combination thereof.

In some embodiments, the intraosseous access system includes where the processing unit includes a network communications logic to provide wired or wireless communication with the external computing device or network about the intraosseous access system progress.

Also disclosed is a method of accessing to an internal cavity including in some embodiments, providing an intraosseous access system including a driver including an access assembly, a motor, and an energy source, a sensor configured to detect an input from one of the motor or energy source, and a processing unit, communicatively coupled with the sensor and configured to receive a first input and second input, detecting a first input, detecting a second input, determining one of the second input being less than the first input, or the first input is less than a threshold value, and modifying operation of one of the motor the energy source.

In some embodiments, the method includes where modifying operation of the motor includes stopping rotation of the motor.

In some embodiments, the method includes where modifying operation of the energy source includes stopping electrical current to the motor.

In some embodiments, the method further includes notifying the user operation of the motor and energy source has been modified.

In some embodiments, the method includes where notifying the user includes illuminating of an LED, vibrating of the IO device, broadcasting an auditory signal or a combination thereof.

In some embodiments, the method includes where notifying the user includes transmitting a notification message over a wired or wireless network including Bluetooth, Wifi, Near Field Communication (NFC), cellular Global System for Mobile Communication (“GSM”), or a combination thereof.

Also disclosed herein is a method of accessing an internal cavity including providing an intraosseous access system including a driver including an access assembly, a motor, and an energy source; a sensor configured to detect an electrical measurement, and a processing unit, communicatively coupled with the sensor detecting a first electrical measurement, determining whether the first electrical measurement is less than a threshold value, and modifying operation of one of the motor the energy source.

In some embodiments, the method includes where the sensor is configured to detect an electrical measurement from one of the energy source or the motor.

In some embodiments, the method includes where the electrical measurement is electrical current.

In some embodiments, the method includes where the threshold value is a predetermined value.

In some embodiments, the method includes where the threshold value can be derived by the processing unit from one or more of the electrical measurements.

In some embodiments, the method further includes a second sensor configured to detect a mechanical measurement.

In some embodiments, the method includes where the mechanical measurement includes torque or rotational speed.

These and other features of the concepts provided herein will become more apparent to those of skill in the art in view of the accompanying drawings and following description, which describe particular embodiments of such concepts in greater detail.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates an exploded view of an embodiment of an intraosseous access medical device system, wherein an access assembly subset of the system is depicted slightly enlarged and in elevation, and an automated driver component is depicted in perspective, in accordance with some embodiments herein.

FIG. 2A illustrates a perspective view of an embodiment of an intraosseous driver, in accordance with some embodiments.

FIG. 2B illustrates a block diagram depicting various elements of an intraosseous device including a sensor and processing unit, in accordance with some embodiments.

FIG. 2C illustrates a perspective view of an embodiment of an intraosseous driver, in accordance with some embodiments.

FIG. 2D illustrates a block diagram depicting various elements of an intraosseous device, in accordance with some embodiments.

FIG. 3 illustrates an exemplary method of using an intraosseous access system to access an internal cavity, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a needle disclosed herein includes a portion of the needle intended to be near a clinician when the needle is used on a patient. Likewise, a “proximal length” of, for example, the needle includes a length of the needle intended to be near the clinician when the needle is used on the patient. A “proximal end” of, for example, the needle includes an end of the needle intended to be near the clinician when the needle is used on the patient. The proximal portion, the proximal-end portion, or the proximal length of the needle can include the proximal end of the needle; however, the proximal portion, the proximal-end portion, or the proximal length of the needle need not include the proximal end of the needle. That is, unless context suggests otherwise, the proximal portion, the proximal-end portion, or the proximal length of the needle is not a terminal portion or terminal length of the needle.

With respect to “distal,” a “distal portion” or a “distal-end portion” of, for example, a needle disclosed herein includes a portion of the needle intended to be near or in a patient when the needle is used on the patient. Likewise, a “distal length” of, for example, the needle includes a length of the needle intended to be near or in the patient when the needle is used on the patient. A “distal end” of, for example, the needle includes an end of the needle intended to be near or in the patient when the needle is used on the patient. The distal portion, the distal-end portion, or the distal length of the needle can include the distal end of the needle; however, the distal portion, the distal-end portion, or the distal length of the needle need not include the distal end of the needle. That is, unless context suggests otherwise, the distal portion, the distal-end portion, or the distal length of the needle is not a terminal portion or terminal length of the needle.

In the following description, certain terminology is used to describe aspects of the invention. For example, in certain situations, the term “logic” is representative of hardware, firmware or software that is configured to perform one or more functions. As hardware, logic may include circuitry having data processing or storage functionality. Examples of such circuitry may include, but are not limited or restricted to a hardware processor (e.g., microprocessor with one or more processor cores, a digital signal processor, a programmable gate array, a microcontroller, an application specific integrated circuit “ASIC,” etc.), a semiconductor memory, or combinatorial elements.

Alternatively, logic may be software, such as executable code in the form of an executable application, an Application Programming Interface (API), a subroutine, a function, a procedure, an applet, a servlet, a routine, source code, object code, a shared library/dynamic load library, or one or more instructions. The software may be stored in any type of a suitable non-transitory storage medium, or transitory storage medium (e.g., electrical, optical, acoustical or other form of propagated signals such as carrier waves, infrared signals, or digital signals). Examples of non-transitory storage medium may include, but are not limited or restricted to a programmable circuit; semiconductor memory; non-persistent storage such as volatile memory (e.g., any type of random access memory “RAM”); or persistent storage such as non-volatile memory (e.g., read-only memory “ROM,” power-backed RAM, flash memory, phase-change memory, etc.), a solid-state drive, hard disk drive, an optical disc drive, or a portable memory device. As firmware, the executable code may be stored in persistent storage.

As used herein, the term “electrical measurement”, can be a quantitative measurement of electricity including electrical current measured in amps, electrical resistance measured in ohms, electrical potential measured in volts, electrical power measured in watts, or the like.

Unless defined otherwise, all technical and scientific terms used herein have the same meaning as commonly understood by those of ordinary skill in the art.

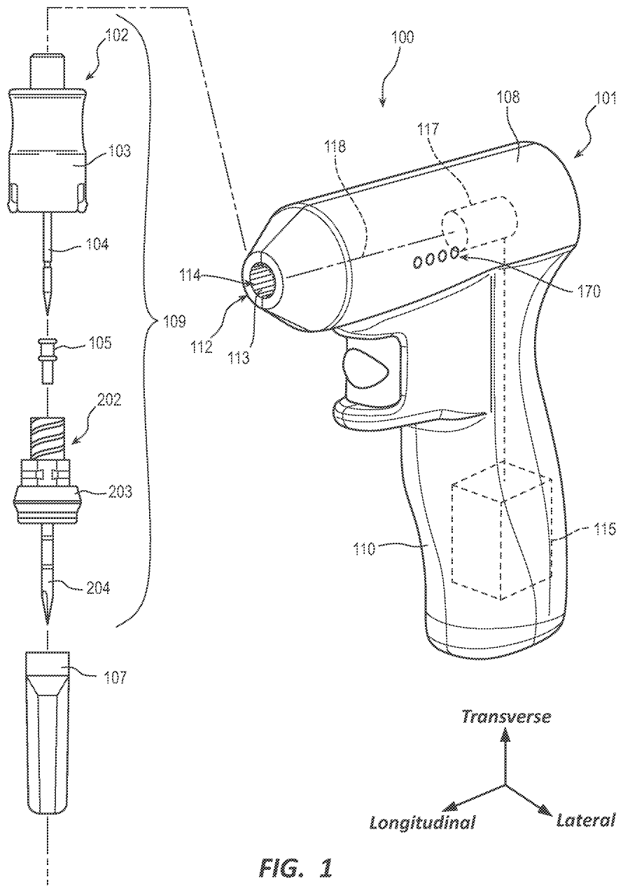

The present disclosure relates generally to intraosseous (“IO”) access device systems that includes one or more sensors, processors or logic, e.g. input receiving logic, threshold logic, motor control logic, energy source logic, or the like, configured to detect access to a medullary cavity. FIG. 1A shows an exploded view of an exemplary intraosseous access system (“system”) 100, with some components thereof shown in elevation and another shown in perspective. In an embodiment, the intraosseous access system 100 can be used to penetrate skin surface tissue layers and underlying hard bone, i.e. bone cortex, for intraosseous access, such as, for example to access the marrow of the bone and/or a vasculature of the patient via a pathway through an interior of the bone, i.e. the medullary cavity. As used herein, an “access event” includes accessing the medullary cavity with an intraosseous access system 100.

In an embodiment, the system 100 includes a driver 101 and an access assembly 109. The driver 101 can be used to rotate the access assembly 109 and “drill” a needle 204 into the bone of a patient. In embodiments, the driver 101 can be automated or manual. As shown in FIG. 1, the driver 101 is an automated driver 101. For example, the automated driver 101 can be a drill that achieves high rotational speeds. In an embodiment, the intraosseous access system 100 can further include an obturator assembly 102, a safety shield (“shield”) 105, and a needle assembly 202, which may be referred to, collectively, as the access assembly 109. The needle assembly 202 can include an access needle (“needle”) 204 supported by a needle hub 203. In an embodiment, the obturator assembly 102 includes an elongate obturator body (“obturator”) 104. As used herein, an obturator 104 includes an elongate medical device configured to be disposed within a lumen of a needle and to prevent bone fragments, tissue, or the like from entering the needle lumen. Advantageously, the obturator 104 prevents tissues from obstructing a fluid flow through the needle lumen, after the needle 204 has been placed to access the medullary cavity. As will be appreciated, in some embodiments, the obturator 104 may be replaced with a different elongated medical instrument. As used herein, the term “elongated medical instrument” is a broad term used in its ordinary sense that includes, for example, such devices as needles, cannulas, trocars, obturators, stylets, and the like. Accordingly, the obturator assembly 102 may be referred to more generally as an elongated medical instrument assembly. In like manner, the obturator 104 may be referred to more generally as an elongated medical instrument.

In an embodiment, the obturator assembly 102 includes a coupling hub 103 that is attached to the obturator 104 in any suitable manner (e.g., one or more adhesives or overmolding). The coupling hub 103 can be configured to interface with the driver 101. The coupling hub 103 may alternatively be referred to as an obturator hub 103 or, more generally, as an elongated instrument hub 103. In an embodiment, the shield 105 is configured to couple with the obturator 104 to prevent accidental needle stick injuries when the obturator 104 is removed after placement of the needle 204.

In an embodiment, the needle assembly 202 includes a needle 204. However, in some embodiments, the needle 204 may be replaced with a different instrument, such as, for example, a cannula, a tube, or a sheath, and/or may be referred to by a different name, such as one or more of the foregoing examples. Accordingly, the needle assembly 202 may be referred to more generally as a cannula assembly or as a tube assembly. In like manner, the needle 204 may be referred to more generally as a cannula. In an embodiment, the needle assembly 202 includes a needle hub 203 that is attached to the needle 204 in any suitable manner. The needle hub 203 can be configured to couple with the obturator hub 103 and may thereby be coupled with the driver 101. The needle hub 203 may alternatively be referred to as a cannula hub 203. In an embodiment, a cap 107 may be provided to cover at least a distal portion of the needle 204 and the obturator 104 prior to use of the access assembly 109. For example, in an embodiment, a proximal end of the cap 107 can be coupled to the obturator hub 103.

In some embodiments, the intraosseous driver 101 can include an energy source 115. In some embodiments, the energy source 115 is configured to energize the rotational movement of a coupling interface 112 and provide a motive force. In some embodiments, the energy source 115 may comprise one or more batteries that provide electrical power for the driver 101.

The energy source 115 may be coupled with the coupling interface 112 via electrical coupling 116 including in some embodiments, an electrical motor that generates mechanical movement from electrical energy provided by an electrical energy source 115. The driver 101 can further include a gear assembly 117 configured to translate rotational movement of the electrical motor 116 to rotational movement of the coupling interface 112 and access assembly 109 coupled thereto.

Further details and embodiments of the intraosseous access system 100 can be found in WO 2018/075694, WO 2018/165334, WO 2018/165339, and US 2018/0116693, each of which is incorporated by reference in its entirety into this application.

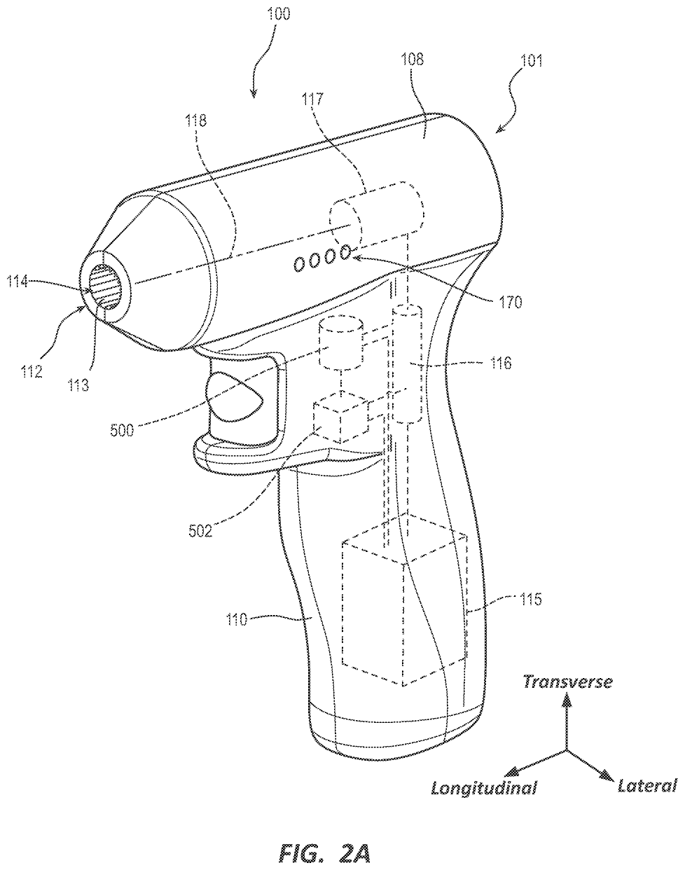

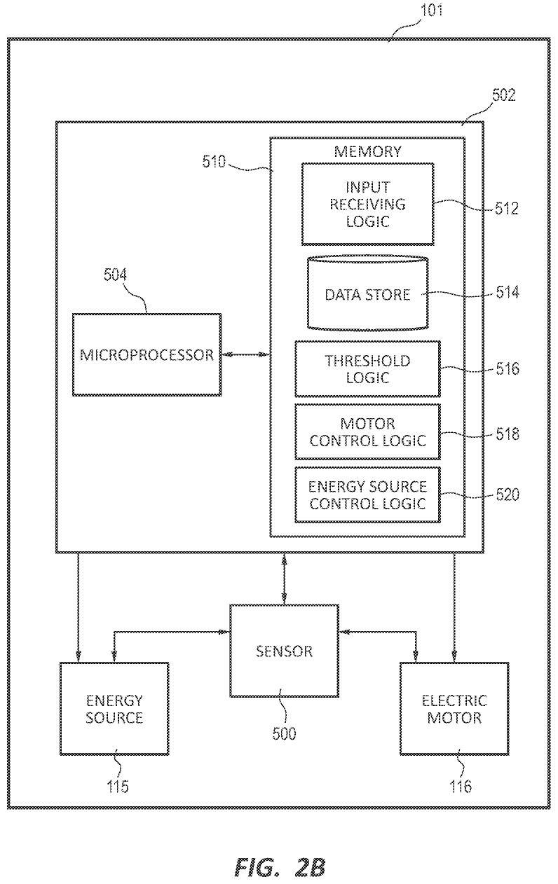

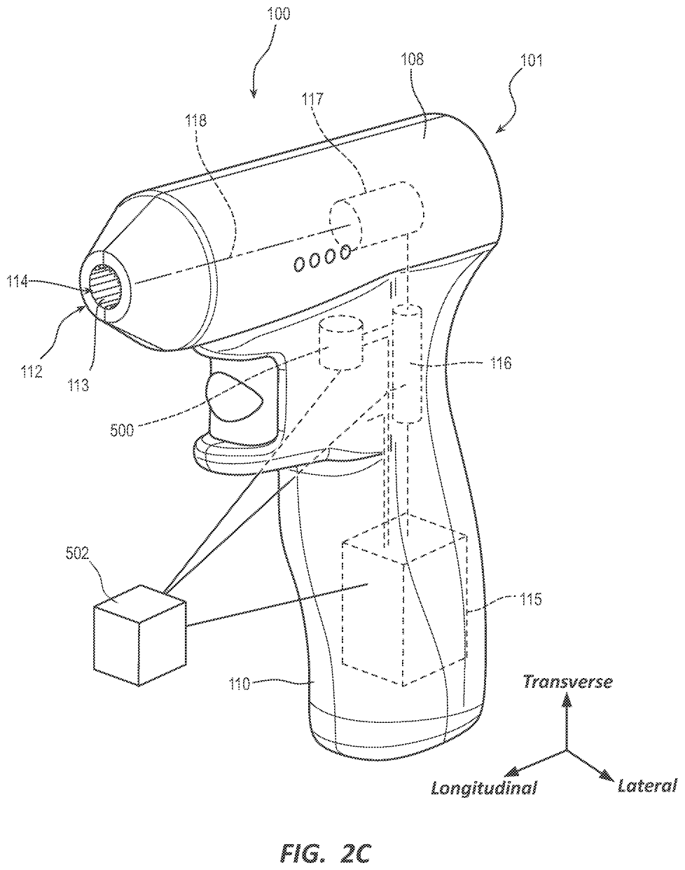

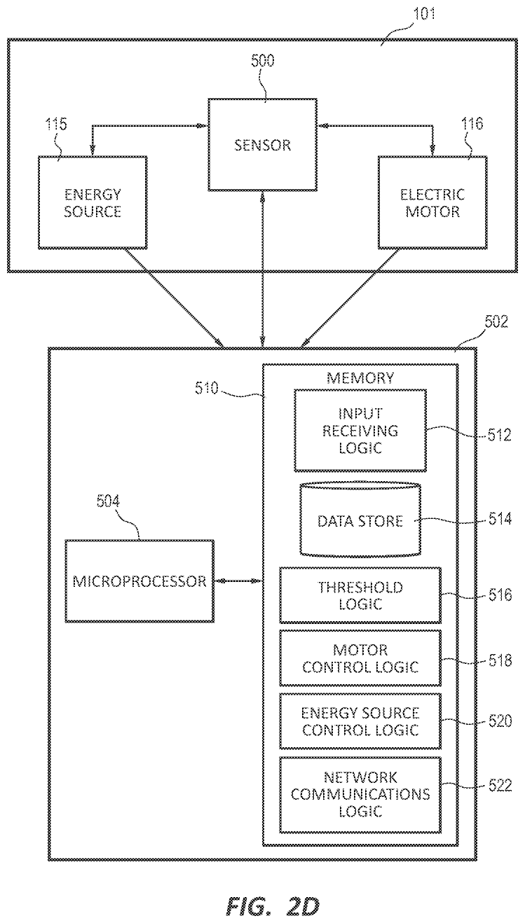

FIG. 2A illustrates a perspective view of an embodiment of an intraosseous access system 100, including a driver 101, in accordance with some embodiments. The intraosseous driver 101 can include one of a sensor 500 or a processing unit 502 that are communicatively coupled to one of the energy source 115 or the electric motor 116, or combinations thereof. For example, in some embodiments, the sensor 500 or the processing unit 502 can be in wired or wireless communication with the energy source 115 or the electric motor 116. In an embodiment, the sensor 500 can include a sensor array including one or more sensors each configured to detect one or more inputs from the system 100.

In some embodiments, the sensor 500 is configured to detect one or more inputs that can be the same or different modalities. Exemplary input modalities can include electrical measurement modalities, e.g. amps, ohms, volts, etc., of either the energy source 115 or the motor 116, or mechanical measurement modalities e.g. torque, rotational speed, etc. of the motor 116. In some embodiments, the sensor 500 includes an ammeter, ohmmeter, or voltmeter for detecting electrical measurements, a torque meter or tachometer to detect a mechanical measurement of the electrical motor 116, combinations thereof, or the like.

In some embodiments, the sensor 500 can begin detecting an input, e.g. an electrical current from the energy source 115, when the electric motor 116 is actuated and draws an electrical current from the energy source 115. In an embodiment, the sensor 500 can detect an input, e.g. the electrical current, torque, etc., continually. In an embodiment, the sensor 500 can continually detect the electrical current draw input without stopping as long as the electric motor 116 draws an electric current from the energy source 115. In some embodiments, the sensor 500 can detect an input, at predetermined time intervals, e.g. 1 second, 1 millisecond, 1 microsecond, etc. however, greater or lesser time intervals are also contemplated.

Once the sensor 500 detects an input, the sensor 500 can communicate the input to a processing unit 502. In some embodiments, the sensor 500 can continually communicate an input to the processing unit 502. In an embodiment, the sensor 500 can communicate an input to the processing unit 502 in response to a trigger, such as when the electric motor 116 is actuated, or similar action. In some embodiments, the sensor 500 can communicate the input to the processing unit 502 at a predetermined time interval, e.g. 1 second, 1 millisecond, 1 microsecond, etc. however, greater or lesser time intervals are also contemplated.

In some embodiments, the sensor 500 can detect and communicate multiple inputs from multiple modalities, e.g. a first input and a second input, to the processing unit 502. In some embodiments, the first input and the second input can be the same or different modalities. In an embodiment, the first input and the second input can be communicated simultaneously, or sequentially. For example, a first input can occur when the electric motor 116 is actuated and the second input can occur at any point in time after the first input.

The driver 101 further includes a processing unit 502 that can be communicatively coupled with one of the sensor 500, the electric motor 116 or the energy source 115. In some embodiments, the processing unit 502 is in wired communication with one of the sensor 500, the electric motor 116 or the energy source 115. In some embodiments, the sensor 500 and processing unit 502 can be communicatively coupled by way of wireless communication. In some embodiments, the processing unit 502 can be in wireless communication with one of the electric motor 116 or the energy source 115. Exemplary wireless communication modalities can include WiFi, Bluetooth, Near Field Communications (NFC), electromagnetic (EM), radio frequency (RF), combinations thereof, or the like.

In some embodiments, the sensor 500 is communicative coupled to the processing unit 502 and provides one or more inputs to the processing unit 502. In some embodiments, the one or more inputs provided to the processing unit 502 by the sensor 500 can include one or more electrical current draw inputs, one or more torque inputs, or the like. In some embodiments, the processing unit 502 receives an input from the sensor 500 and can modify the operation of one of the electric motor 116 or operation of the energy source 115, as will be described in more detail herein.

As illustrated in FIG. 2B, in some embodiments, the processing unit 502 can include one or more processor(s), storage, communications logic, control logic, or the like. The processing unit 502 can be configured to receive information from the sensor 500 to determine if the needle 204 has accessed the medullary cavity, and modify the activation of the driver 101. In some embodiments, the processing unit 502 includes a microprocessor 504 and is coupled to memory 510. In some embodiments, the processing unit 502 is configured to receive an input communicated thereto by the sensor 500. In some embodiments, the processing unit 502 can be constantly sampling the sensor 500 for an input.

In an embodiment, the processing unit 502 can compare a first input, e.g. a first electrical current draw value, and a second input, e.g. a second electrical current draw value to determine if the medullary cavity has been accessed. In an embodiment, the processing unit 502 can compare an input with a threshold value to determine if the medullary cavity has been accessed. In some embodiments, the processing unit 502 can modify operation of the electric motor 116 based on either a relative change between the first input and the second input, or by comparing the input with a threshold. In an embodiment, the threshold can be a predetermined value or can be derived by the processing unit 502, e.g. by the threshold logic 516 from one or more inputs.

In some embodiments, the processing unit 502 is coupled to memory 510 that includes an input receiving logic 512, a data store 514, the threshold logic 516, an electrical motor control logic 518 and an energy source control logic 520. The input receiving logic 512 can receive an input from the sensor 500. The data store 514 stores the input values received from the sensor 500, e.g. electrical measurement values, mechanical measurement values, or the like, from the sensor 500. The threshold logic 516 can store or derive a threshold value and compare the input with the threshold value. In some embodiments, the threshold logic 516 can store/derive one or more threshold values calibrated for different tissues. For example, different tissues require different electrical current draws or torque to drive the needle 204 through the various tissues. Penetrating the bone cortex would require the most torque from the motor 116 and therefore more electrical current draw from the energy source 115. Whereas skin, muscle, or tissues within the medullary cavity would require less torque and therefore less electrical current draw.

In some embodiments, the threshold value can be calibrated relative to the individual patient, for example to normalize for differences in age sex, health condition, or the like. In some embodiments, the threshold can be an absolute value provided by the user or preprogramed to the processing unit 502 during manufacture. In some embodiments, the threshold value can be derived from information provided by the user, e.g. details about the patient, age, gender, health condition, etc. In some embodiments, the threshold can be derived from one or more inputs from the system 100 during operation, e.g. by comparing a first input and a second input. The motor control logic 518, in some embodiments, can be configured to modify the operation of the electric motor 116. The energy source control logic 520, in some embodiments, can be configured to modify the energy provided from the energy source 115 to the electric motor 116.

In some embodiments, the processing unit 502 can automatically stop actuation of the electric motor 116, indicating to the user that the medullary cavity has been accessed, and/or can prevent backwalling. In some embodiments, if the operation of the driver 101 is modified, the processing unit 502 can provide a notification to the user that a modification in one of the energy source 115 or the electric motor 116 has occurred. In some embodiments, the notification can include a visual, tactile, or auditory notification, or combinations thereof, for example an illumination of a light-emitting diode, vibration of the driver 101, an auditory signal, or the like. Advantageously, the notification can clearly indicate to the user that the medullary cavity has been accessed.

In an exemplary method of use, an intraosseous access system 100 is provided, as described herein and can automatically determine access to the medullary cavity and modify the actuation of the driver 101. The driver 101 can include an automatic driver that uses an energy source 115, e.g. a battery, and an electric motor 116 to rotate the access assembly 109 and drill a needle 204 into the bone of a patient. The electric motor 116 draws an electrical current from the energy source 115 and provides a rotational torque to the access assembly 109 and needle 204 coupled thereto. In some embodiments, a greater amount of torque is required to drive the needle 204 through more dense tissues, such as bone, relative to softer tissues such as tissues disposed within the medullary cavity. As such, a greater draw of electrical current is required to provide a greater amount of torque.

In an embodiment, as the driver 101 is actuated and penetrates a skin surface of the patient, a first torque amount is required to penetrate the skin and associated tissues, which draws a first electrical current draw value. A first input, e.g. torque or electrical current draw, is detected by the sensor 500 and communicated to the processing unit 502. When the needle 204 penetrates into the bone cortex, a second input, e.g. torque or electrical current draw value, is measured by the sensor 500 and monitored by the processing unit 502. The difference between a torque required to penetrate the skin, and a torque required to penetrate the bone cortex requires an increase in electrical current draw to maintain the rotational speed of the access assembly 109 and maintain penetration of the bone including the bone cortex and medullary cavity. In some embodiments, the first electrical current draw value can be when the needle 204 penetrates the skin, and the second electrical current draw value can be when the needle 204 penetrates the bone cortex. In some embodiments, the first electrical current draw value can be when the needle 204 is rotating freely in ambient conditions, and the second electrical current draw value can be when the needle 204 penetrates the skin tissues.

In an embodiment, as the needle 204 penetrates the bone cortex, a first input, e.g. electrical current draw or torque, is required to maintain the rotational speed of the access assembly 109 and maintain penetration of the bone. As the needle 204 accesses the medullary cavity, the torque demands of the motor 116 and/or electrical demands decreases, providing a second input that is less than the first input or less than a threshold value. The processing unit 502 can determine the second input is less than the first input, or less than a threshold value, and modify the operation of the driver 101.

In an embodiment, the threshold logic 516 can provide either a predetermined threshold value or can determine a threshold value based on the previous electrical current draw information, e.g. from measuring electrical current draw values as the needle 204 penetrates the skin surface and bone cortex. The processing unit 502 can then determine access to the medullary cavity by comparing a change in inputs, e.g. electrical draws, between a first input and a second input, or a drop in input value across a threshold value. The motor control logic 518 can then modify the actuation of the electric motor 116. In an embodiment, the energy source control logic 520 can modify an electrical current draw from the energy source 115. Further, the processor unit 502 may notify the user that the operation of the driver 101 has been modified, by one or more notifications, as described herein. Advantageously, the driver 101 can automatically calibrate to different bone densities based on inputs detected during penetration of the skin, muscle, or bone cortex tissues.

In an embodiment, if the user withdraws the driver 101 before accessing the medullary cavity, the actuation of the electric motor 116 can be automatically stopped based on a change in input. Advantageously, this can provide a safety mechanism should the driver 101 be withdrawn prematurely. The driver 101 can then be restarted through actuation of the electric motor 116. In an embodiment, if the user reduces the rotational speed of the electric motor 116 and thus the electrical current draw on the energy source 115, the actuation of the electric motor 116 or energy source 115 can be stopped.

As shown in FIGS. 2C-2D, in some embodiments, the processor unit 502, or components thereof, may be disposed remotely from the driver 101. In this embodiment, the processor unit 502 can be in wired or wireless communication with one of the sensor 500, the energy source 115, or the electric motor 116 of the driver 101. The processor unit 502 can then receive input(s), and modify an electric motor 116 or energy source 115, as described herein.

Advantageously when the processing unit 502 is disposed remotely from the driver 101, the processing unit 502 may be disposed in a charging station or base station disposed nearby. In some embodiments, the processing unit 502 can be communicatively coupled with one or more external computing devices, or with a centralized or decentralize network, or combinations thereof. In some embodiments, the external computing device includes one of an external monitor, laptop, computer, mobile device, smart phone, tablet, “wearable” electronic device, centralized network server, decentralized network server, a hospital intranet server, an Electronic Health Record (“EHR”) system, a “cloud” based network server, or an internet server. The processing unit 502 can include a network communications logic 522 that can provide wired or wireless communication with the external computing device or network. Exemplary wireless communication can include Bluetooth, Wifi, Near Field Communication (NFC), cellular Global System for Mobile Communication (“GSM”), combinations thereof, or the like.

In some embodiments, the processing unit 502 can immediately relay information about the access event to one or more external computing devices, other medical devices, centralized or decentralized networks or the like, by way of a network communication logic 522. Advantageously, relaying information about the access event would allow clinicians to monitor the medullary access event progress and provide situational awareness, e.g. to a hospital team. In some embodiments, the processing unit 502 may be synchronized with other medical devices or may automatically update a patient's electronic health record when a medullary cavity access event is completed.

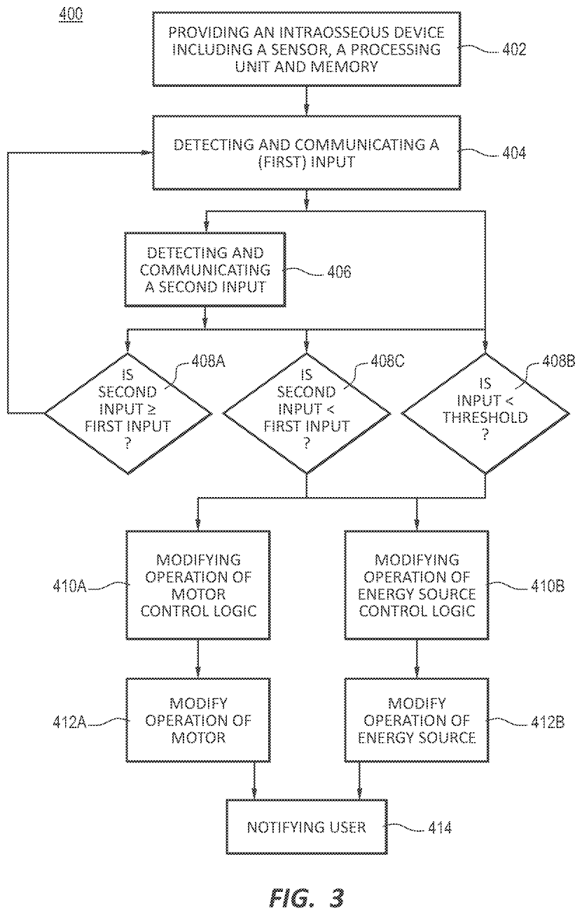

FIG. 3 illustrates an exemplary method of using an intraosseous access system 100 to access a medullary cavity, as described herein. The first step (block 402) in the method 400 includes providing an intraosseous access system 100 including an access assembly 109 coupled to an intraosseous driver 101. In some embodiments, the driver 101 includes an electric motor 116, an energy source 115, a sensor 500, and a processing unit 502 including memory 510, each of which can be communicatively coupled with each other.

The second step (block 404) includes the sensor 500 detecting and communicating a first input. In some embodiments, the first input includes a first electrical current draw value from one of the electric motor 116 or the energy source 115. In some embodiments, the first input can be a mechanical measurement, e.g. torque, of the motor 116.

Optionally, a third step (block 406) includes the sensor 500 detecting and communicating a second input. In some embodiments, the second input includes a second electrical current draw value from one of the electric motor or the energy source 115, or a second mechanical measurement, e.g. torque, of the motor 116.

The fourth step can include comparing the either the first input or the second input relative to a threshold value to determine if the input has dropped below a threshold (diamond 408B), determining if the second input is greater than or equal to the first input (diamond 408A), or determining if the second input decreased from the first (diamond 408C). In some embodiments, the second input includes a second electrical current draw value from one of the electric motor 116 or the energy source 115. If the second input is greater than or equal to the first input, the method 400 returns to the second step (block 404). In some embodiments, the threshold can be predetermined or calibrated for particular cohort of patients, or the system can derive the threshold based on information from the user or from previous inputs to the system.

If the second input is less than the first input or if one of the first input or the second input is below the threshold, thereby indicating that the intraosseous access system 100 has accessed the medullary cavity, the method 400 moves to the fifth step of modifying operation of the motor control logic (block 410A) and/or modifying operation of energy source control logic (block 410B). The method 400 continues further to the sixth step of modifying operation of the motor (block 412A) and/or modifying operation of energy source (block 412B) respectively. In some embodiments, modifying operation of the motor includes stopping the rotation of the electric motor 116. In some embodiments, modifying operation of the energy source (block 412B) includes ceasing electrical current from the energy source 115 to the motor. In an embodiment, the method 400 can move from modifying operation of motor control logic (block 410A) to modifying operation of the motor (block 412A) independently of blocks 410B and 412B. In an embodiment, the method 400 can move from modifying operation of energy source control logic (block 410B) to modifying operation of the energy source (block 412B) independently of blocks 410A and 412A. In an embodiment, the method 400 can simultaneously move from modifying the operation of the motor control logic (block 410A) to modifying the operation of the motor (block 412A) while moving from modifying the operation of the energy source control logic (block 410B) to modifying operation of the energy source (block 412B).

In an embodiment, the last step 412 includes notifying the user that one of the motor operation, or the energy source have been modified. In some embodiments, the notifying the user includes illumination of an LED, vibration of the IO device, an auditory signal or a combination thereof. In some embodiments, notifying the user includes transmitting a notification message over a wired or wireless network including Bluetooth, Wifi, Near Field Communication (NFC), cellular Global System for Mobile Communication (“GSM”), combinations thereof, or the like. In some embodiments, notifying the user includes transmitting a message to an external computing device including one of an external monitor, laptop, computer, mobile device, smart phone, tablet, “wearable” electronic device, centralized network server, decentralized network server, a hospital intranet server, an Electronic Health Record (“EHR”) system, a “cloud” based network server, or an internet server.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. An intraosseous access system, comprising:
a driver including an access assembly, a motor, and an energy source;
a sensor configured to detect a first input from one of the motor or the energy source; and
a processing unit communicatively coupled with the sensor, the processing unit configured to (i) receive the first input from the sensor, (ii) determine access to a medullary cavity, and (iii) modify operation of one of the motor or the energy source.

2. The intraosseous access system according to claim 1, wherein the processing unit compares a second input relative to the first input to determine access to a medullary cavity.

3. The intraosseous access system according to claim 1, wherein the processing unit compares the first input relative to a threshold value to determine access to a medullary cavity.

4. The intraosseous access system according to claim 1, wherein the sensor is configured to detect one of an electrical measurement or a mechanical measurement.

5. The intraosseous access system according to claim 4, wherein the sensor includes one of an ammeter, ohmmeter, voltmeter, torque meter, or tachometer.

6. The intraosseous access system according to claim 1, wherein the sensor and the processing unit are located within the driver.

7. The intraosseous access system according to claim 1, wherein the sensor is located within the driver and the processing unit is located remotely from the driver and is in wireless communication with one of the sensor, the motor or the energy source.

8. The intraosseous access system according to claim 3, wherein the threshold value is a predetermined value.

9. The intraosseous access system according to claim 3, wherein the threshold value can be derived by the processing unit relative to one of the first input or a second input.

10. The intraosseous access system according to claim 3, wherein the threshold value can be calibrated for electrical current draws required to drive the intraosseous access system through tissues to access the medullary cavity.

11. The intraosseous access system according to claim 3, wherein the threshold value can be normalized for an age, sex, or health condition of a patient.

12. The intraosseous access system according to claim 1, wherein the processing unit includes a network communications logic to provide wired or wireless communication with the external computing device or network.

13. A method of accessing an internal cavity, comprising:
providing an intraosseous access system comprising:
a driver including an access assembly, a motor, and an energy source;
a sensor configured to detect an input from one of the motor or the energy source; and
a processing unit, communicatively coupled with the sensor;
detecting a first input;
detecting a second input;
determining whether the second input is less than the first input, or the first input is less than a threshold value; and
modifying operation of one of the motor the energy source.

14. The method according to claim 13, wherein modifying operation of the motor includes stopping rotation of the motor.

15. The method according to claim 13, wherein modifying operation of the energy source includes stopping electrical current to the motor.

16. The method according to claim 13, further including notifying the user that the operation of the motor and energy source has been modified.

17. The method according to claim 16, wherein notifying the user includes illuminating of an LED, vibrating of the TO device, or broadcasting an auditory signal.

18. The method according to claim 16, wherein notifying the user includes transmitting a notification message over a wired or wireless network.

19. The method according to claim 18, wherein the wireless network includes Bluetooth, Wifi, Near Field Communication (NFC), or cellular Global System for Mobile Communication (“GSM”).

20. A method of accessing an internal cavity, comprising:
providing an intraosseous access system comprising:
a driver including an access assembly, a motor, and an energy source;
a sensor configured to detect an electrical measurement; and
a processing unit, communicatively coupled with the sensor;
detecting a first electrical measurement;
determining whether the first electrical measurement is less than a threshold value; and
modifying operation of one of the motor the energy source.

21. The method according to claim 20, wherein the sensor is configured to detect an electrical measurement from one of the energy source or the motor.

22. The method according to claim 21, wherein the electrical measurement is electrical current.

23. The method according to claim 20, wherein the threshold value is a predetermined value.

24. The method according to claim 20, wherein the threshold value can be derived by the processing unit from one or more of the electrical measurements.

25. The method according to claim 20, further including a second sensor configured to detect a mechanical measurement.

26. The method according to claim 25, wherein the mechanical measurement includes torque or rotational speed.
